(19) United States
(12) Patent Application Publication
(10) Pub. No.: US 2013/0296712 A1
(43) Pub. Date: Nov. 7, 2013

(54) INTEGRATED NON-CONTACT DIMENSIONAL METROLOGY TOOL

(71) Applicant: COVIDIEN LP, Mansfield, MA (US)

(72) Inventor: Ravi Shankar Durvasula, Cheshire, CT (US)

(73) Assignee: Covidien LP, Mansfield, MA (US)

(21) Appl. No.: 13/865,380

(22) Filed: Apr. 18, 2013

Related U.S. Application Data
(60) Provisional application No. 61/641,968, filed on May 3, 2012.

(51) Int. Cl. A61B 5/00; A61B 1/04; A61B 1/06

(52) U.S. Cl. CPC A61B 5/0084; A61B 1/0684; A61B 1/04; A61B 5/0062
USPC 600/477

(57) ABSTRACT

An apparatus for determining endoscopic dimensional measurements, including a light source for projecting light patterns on a surgical sight including shapes with actual dimensional measurements and fiducials, and a means for analyzing the projecting light patterns on the surgical sight by comparing the actual dimensional measurements of the projected light patterns to the surgical site. The projected light patterns may include multiple wavelengths of light for measurements of different features of tissue and may be produced using a laser in conjunction with a light shaping optical diffuser, or using a light emitting diode in conjunction with a light shaping optical diffuser, or using a special filter. The projected light patterns may take the form of concentric rings with each ring representing a radius of a given dimension and may be a collimated pattern which does not significantly change size as a function of a distance to a projected plane.

### CROSS REFERENCE TO RELATED APPLICATION

The present application claims the benefit of and priority to U.S. Provisional Application Ser. No. 61/641,968, filed on May 3, 2012, the entire contents of which are incorporated herein by reference.

### BACKGROUND

1. Technical Field

The present disclosure relates to a method and apparatus for measuring a dimension of a target site. More particularly, the present disclosure relates to a method and apparatus for projecting a pattern of a known size onto a target site for measuring a desired portion of the target site.

2. Background of the Related Art

Minimally invasive surgery, e.g., laparoscopic, endoscopic, and thoroscopic surgery, has many advantages over traditional open surgeries. In particular, minimally invasive surgery eliminates the need for a large incision, thereby reducing discomfort, recovery time, and many of the deleterious side effects associated with traditional open surgery.

The minimally invasive surgeries are performed through small openings in a patient's skin. These openings may be incisions in the skin or may be naturally occurring body orifices (e.g., mouth, anus, or vagina). In general, an insufflation fluid is used to enlarge the area surrounding the target surgical site to create a larger, more accessible work area.

In many surgical situations, having real-time metrology tools providing dimensional measurements would be helpful for surgeons. This is especially the case in minimally invasive surgery where access to the surgical site is limited. The tools can either be stand alone tools or be integrated with surgical instruments. While the size of the metrology tool in most open surgical applications is not as critical, for minimally invasive procedures, it would be helpful to have as small of a form factor as possible.

Both due to accuracy considerations and due to the complex topographies of the surgical site and the need to keep the site as sterile as possible, it would be ideal for the metrology tools to operate in a non-contact fashion.

### SUMMARY

The current disclosure describes several embodiments of endoscopic metrology tools which can be realized in a small form factor and employ non-contact methods for dimensional measurements. These embodiments primarily exploit optical and/or acoustical methods.

An aspect of the present disclosure provides a method of measuring a dimension of a target site which includes projecting light patterns on a surgical sight from a light source and analyzing the projected light patterns on the surgical sight by comparing the actual dimensional measurements of the projected light patterns to the surgical site. The light patterns may include shapes with actual dimensional measurements and fiducials, and also may include multiple wavelengths of light for measurements of different features of a tissue. The projected light patterns may be produced using a laser in conjunction with a light shaping optical diffuser, or using a light emitting diode in conjunction with a light shaping optical diffuser, or using a special filter. The projected light patterns may take the form of concentric rings with each ring representing a radius of a given dimension. The projected light pattern may be a collimated pattern which does not significantly change size as a function of a distance to a projected plane.

Another aspect of the present disclosure provides a method of measuring a dimension of a target site which includes projecting light patterns on a surgical sight from a light source and analyzing the projected light patterns on the surgical sight by comparing the actual dimensional measurements of the projected light patterns to the surgical site, obtaining a pixelized image from an imaging device wherein the projected fiducials are imaged on to a pixel array sensor of the image, and developing dimensional features of interest in the surgical site based on prior knowledge of relative size or shape of the fiducials. Further, a relative difference may be assessed between a metrology tool and a feature of interest employing triangulation techniques. The triangulation techniques may include a triangulation obtained using a single imaging device, multiple imaging devices, or a combination of imaging device(s) and collimated light sources.

Another aspect of the present disclosure provides an apparatus for determining endoscopic dimensional measurements, including a light source for projecting light patterns on a surgical sight including shapes with actual dimensional measurements and fiducials, and a means for analyzing the projecting light patterns on the surgical sight by comparing the actual dimensional measurements of the projected light patterns to the surgical site. The projected light patterns may include multiple wavelengths of light for measurements of different features of tissue, and may be produced using a laser in conjunction with a light shaping optical diffuser, or using a light emitting diode in conjunction with a light shaping optical diffuser, or using a special filter. The projected light patterns may take the form of concentric rings with each ring representing a radius of a given dimension, and also may be a collimated pattern which does not significantly change size as a function of a distance to a projected plane.

Another aspect of the present disclosure provides the apparatus described above, further including an imaging device which is capable of obtaining a pixelized image. The imaging device may be a CMOS camera or a raster scanning device.

### BRIEF DESCRIPTION OF THE DRAWINGS

The above and other aspects, features, and advantages of the present disclosure will become more apparent in light of the following detailed description when taken in conjunction with the accompanying drawings in which:

FIG. 1 is a side, schematic view of a projector assembly according to the principles of the present disclosure;

FIG. 2 is front, schematic view of the projector assembly of FIG. 1;

FIG. 3 is a side, perspective view of a metrology system according to an embodiment of the present disclosure;

FIG. 4 is a side, schematic view of a metrology system according to another embodiment of the present disclosure;

### DETAILED DESCRIPTION

Particular embodiments of the present disclosure are described hereinbelow with reference to the accompanying drawings; however, it is to be understood that the disclosed embodiments are merely exemplary of the disclosure and may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

Like reference numerals may refer to similar or identical elements throughout the description of the figures. As shown in the drawings and described throughout the following description, as is traditional when referring to relative positioning on a surgical instrument, the term “proximal” refers to the end or portion of the apparatus which is closer to the user and the term “distal” refers to the end or portion of the apparatus which is farther away from the user. The term “clinician” refers to any medical professional (i.e., doctor, surgeon, nurse, or the like) performing a medical procedure involving the use of embodiments described herein.

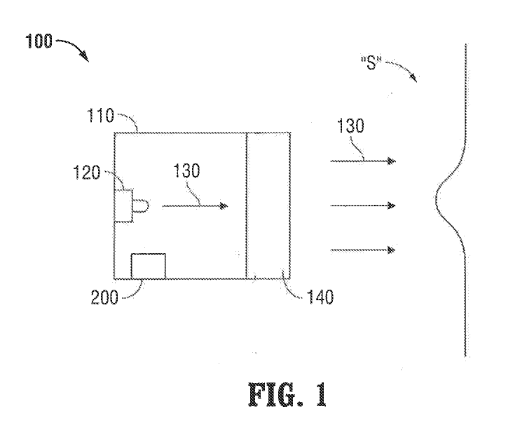

As shown in FIG. 1, metrology system 100 includes a projector assembly 110. Projector assembly 110 includes at least one light emitter 120 such as, for example, LED, laser diode or any combination thereof, and a mask 140. Mask 140 may include a light shaping optical diffuser, a special filter, or any other suitable object. Each light emitter 120 emits a light beam 130 for creating a light pattern on a target site “S.” Adjacent light beams 130 have a fixed distance therebetween. Light beams 130 may be collimated for increased precision of the light pattern. Light beam 130 may be any suitable form of light, such as coherent, partially coherent, visible, infrared, or ultraviolet. Light beam 130 has a wavelength of, for example, 532 nm, to differentiate light beams 130 from a color of any naturally occurring tissue in the human body. Additionally or alternatively, light beams 130 may be multiple wavelengths of light for measurement of different features or for simultaneously outlining margins of diseased tissue. Light emitters 120 are powered by a power source 200 disposed in handle member 200. However, as shown in FIG. 1, it is also envisioned that power source 200 may be disposed within the projector assembly 110. The power source may be a standard commercial battery pack.

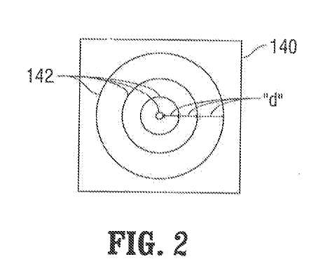

Referring to FIG. 2, mask 140 may be semi-transparent and/or may have a substantially opaque mask pattern 142 thereon. Mask patterns 142 may have markings of known distances therebetween. For example, mask pattern 142 may be a series of uniformly spaced concentric circles. Additionally, or alternatively, the actual dimensions of the known distances “d” may also be projected. It is understood that the pattern may take on multiple shapes and forms.

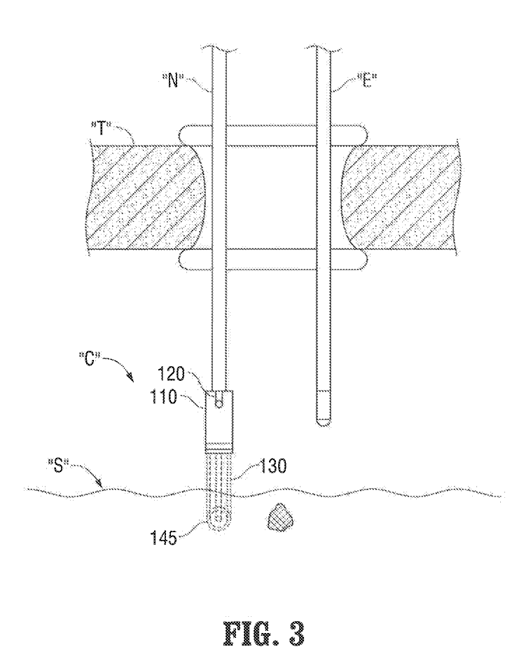

Turning to FIG. 3, a method of use of metrology system 100 is illustrated. As seen in FIG. 3, a target site “S” exists within a cavity “C” under tissue “T”. Metrology system 100 is attached to a distal end of a surgical instrument “N”. Surgical instrument “N” is inserted through a surgical access port “P” positioned in an opening in tissue “T”. An endoscope “E” is inserted through surgical access port “P” for viewing target site “S”.

With continued reference to FIG. 3, light emitter 120 emits light beams 130 to create light pattern 145 on target site “S”. As mentioned above, the light pattern 145 may include actual dimensions of the projected shapes. At this point, a user can view the pattern directly or use an external scope, such as a laparoscope or endoscope “E”, to measure a desired region on the target site “S”. This can be achieved by directing the light pattern 145 directly on the desired region of the target site “S” or on a region adjacent to the desired region of the target site “S”. Directing light pattern 145 directly on the desired region of target site “S” enables a user to view the markings of known distances “d” and directly measure the desired region by viewing the pattern on the desired region or target site “S”.

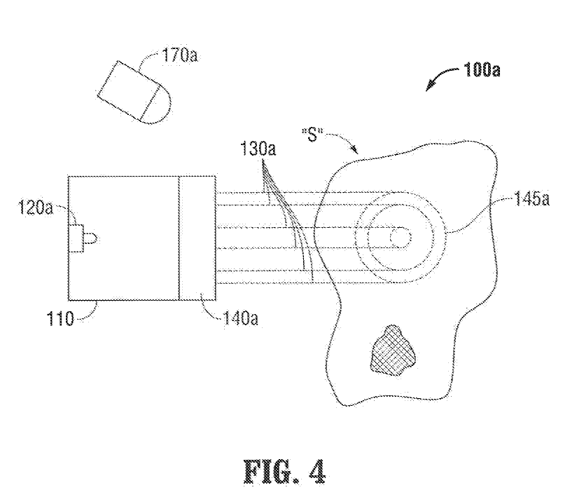

Turning to FIG. 4, a metrology system in accordance with an alternate embodiment of the present disclosure is generally designated as 100a. Metrology system 100a is similar to metrology system 100 and thus will only be discussed as necessary to identify the differences in construction and operation thereof.

Continuing with reference to FIG. 4, metrology system 100a has a projector assembly 110a, at least one light emitter 120a disposed within the projector assembly 110a, mask 140a, and an imaging device 170a. Imaging device 170a is capable of obtaining a pixelized image of target site “S” including light pattern 145a and the desired portion to be measured on target site “S”. Imaging device 170a may be a CMOS camera or a raster scanning device. Imaging device 170a may be disposed within projector assembly 110a or alternatively, may be separate from projector assembly 110a.

With continued reference to FIG. 4, similar to the system described in FIG. 3, light emitter 120a emits lights beams 130a to create light pattern 145a on target site “S”. Specific fiducials can be projected on the surgical site “S” which can be imaged on to a pixel arrayed sensor of the image where based on prior knowledge of the relative size and shape or location of the fiducials, image processing algorithms establish dimensional features of interest on the target site “S”. For additional accuracy, although not shown, metrology may be performed from multiple known relative angles.

Alternatively or additionally, triangulation techniques may be employed to assess the relative distances between the metrology tools and the features of interest. Triangulation could be obtained in multiple ways including using a single imaging device, multiple imaging devices, or a combination of an imaging device(s) and collimated light sources. Alternate optical or acoustical methods can also be employed for range finding. An example of which would be optical or acoustical interferometers. For additional accuracy, metrology may be performed from multiple known relative angles.

As can be appreciated from the foregoing description and drawings, embodiments of an optical metrology and image correction system according to the present disclosure have been described which yield methods for real-time in-body-cavity metrology employing visible, ultraviolet or near-infrared (IR) radiation, which is either coherent or incoherent, to reduce overall surgery time and the cognitive burden on the surgeon. The embodiments also potentially improve patient outcome with more accurate, smaller (depending on the miniaturization scale) incision procedures, which are less prone to human errors or miscalculations.

Improvements in the surgical procedures originate from both savings in time and from more accurate surgical choices by a given surgeon when attempting to choose measurement-dependent devices for a give in-body task or procedure, such as mesh size during a hernia repair.

While several embodiments of the disclosure have been shown in the drawings, it is not intended that the disclosure be limited thereto, as it is intended that the disclosures be as broad in scope as the art will allow and that the specification be read likewise. Therefore, the above description should not be construed as limiting, but merely as exemplifications of particular embodiments.

What is claimed:

1. A non-contacting endoscopic metrology method, comprising the steps of;
projecting light patterns on a surgical sight from a light source, wherein the light patterns comprise shapes with actual dimensional measurements and fiducials; and
analyzing the projected light patterns on the surgical sight by comparing the actual dimensional measurements of the projected light patterns to the surgical site.

2. The method as claimed in claim 1, wherein the projected light patterns include multiple wavelengths of light for measurements of different features of a tissue.

3. The method as claimed in claim 1, wherein the projected light patterns are accomplished using a laser in conjunction with a light shaping optical diffuser.

4. The method as claimed in claim 1, wherein the projected light patterns are accomplished using a light emitting diode in conjunction with a light shaping optical diffuser.

5. The method as claimed in claim 1, wherein the projected light patterns are accomplished using a spatial filter.

6. The method as claimed in claim 1, wherein the projected light pattern is a collimated pattern which does not significantly change size as a function of a distance to a projected plane.

7. The method as claimed in claim 1, further comprising the steps of;
obtaining a pixelized image from an imaging device wherein the projected fiducials are imaged on to a pixel array sensor of the image; and
developing dimensional features of interest in the surgical site based on prior knowledge of relative size or shape of the fiducials.

8. The method as claimed in claim 7, further comprising the step of assessing a relative difference between a metrology tool and a feature of interest employing triangulation techniques.

9. The method as claimed in claim 8, wherein the triangulation techniques comprise a triangulation obtained using a single imaging device.

10. The method as claimed in claim 8, wherein the triangulation techniques comprise a triangulation obtained using multiple imaging devices.

11. The method as claimed in claim 8, wherein the triangulation techniques comprise a triangulation obtained using a combination of an imaging device and collimated light sources.

12. An apparatus for determining endoscopic dimensional measurements, comprising;
a light source for projecting light patterns on a surgical sight wherein the light patterns comprise shapes with actual dimensional measurements and fiducials; and
a means for analyzing the projecting light patterns on the surgical sight by comparing the actual dimensional measurements of the projected light patterns to the surgical site.

13. The apparatus as claimed in claim 13, wherein the projected light patterns include multiple wavelengths of light for measurements of different features of a tissue.

14. The apparatus as claimed in claim 12, wherein the projected light patterns are accomplished using a laser in conjunction with a light shaping optical diffuser.

15. The apparatus as claimed in claim 12, wherein the projected light patterns are accomplished using a light emitting diode in conjunction with a light shaping optical diffuser.

16. The apparatus as claimed in claim 12, wherein the projected light patterns are accomplished using the light source with a spatial filter.

17. The apparatus as claimed in claim 12, wherein the projected light pattern is a collimated pattern which does not significantly change size as a function of a distance to a projected plane.

18. The apparatus as claimed in claim 12, further comprising an imaging device capable of obtaining a pixelized image.

19. The apparatus as claimed in claim 18, wherein the imaging device is a CMOS camera.

20. The apparatus as claimed in claim 18, wherein the imaging device is a raster scanning device.
